(19) United States
(12) Patent Application Publication
(10) Pub. No.: US 2013/0184436 A1
(43) Pub. Date: Jul. 18, 2013

(54) APPARATUS AND METHOD FOR SEMI-AUTOMATED PARALLEL SYNTHESIS OF PEPTIDES

(71) Applicant: Nanjingjinsirui Science & Technology Biology Corporation, Nanjing (CN)

(72) Inventors: Zhuying WANG, Monmouth Junction, NJ (US); Jinyu YANG, Nanjing (CN); Wenyu XU, Nanjing (CN); Tao BAI, Nanjing (CN)

(73) Assignee: Nanjingjinsirui Science & Technology Biology Corporation, Nanjing (CN)

(21) Appl. No.: 13/649,246

(22) Filed: Oct. 11, 2012

(30) Foreign Application Priority Data
Jan. 17, 2012 (CN) 201220019160.7

(51) Int. Cl. B01J 19/18; B01J 19/24; C07K 1/06

(52) U.S. Cl. 530/334; 422/131; 422/135

(57) ABSTRACT

An apparatus is provided for the semi-automated parallel synthesis of multiple peptides. The apparatus includes an array of nozzles, each positioned above or adjacent a separate reaction container, two or more liquid reservoirs, each reservoir coupled to the liquid dispenser(s), and base chamber(s) connected to the reaction containers for removing liquid there from. The addition and removal of liquids may be controlled by programmable electromagnetic valves. The apparatus may also be used for other parallel solid phase reactions. A method is also provided for synthesizing multiple polypeptides or other macromolecules, for example by using the above apparatus, wherein multiple common steps are performed automatically while reactants are added manually.

### BACKGROUND OF THE INVENTION

Peptides are short polymers of amino acids containing peptide bonds, which link the carboxyl group of one amino acid to the amino group of a second amino acid. Peptides are distinguished from proteins on the basis of size, typically containing fewer than 100 monomer units (amino acid units). Peptides are involved in many processes in living organisms. Therefore, as small molecule drugs, antigens, hormones, ligands, vaccines, antibiotics, toxins, etc., peptides are playing more and more important roles in modern biology research. To meet the need of large quantities of peptides, the chemical synthesis of peptides has become an immeasurably valuable tool in the field of scientific research.

Early syntheses of peptides were performed in solution, requiring significant bench work. Solid-phase peptide synthesis (SPPS), pioneered by Robert Bruce Merrifield (J. Am. Chem. Soc. 85 (14): 2149-2154, 1963), resulted in a paradigm shift within the peptide synthesis community. This is now the commonly accepted method for creating peptides in the lab in a synthetic manner. SPPS can be performed either manually or automatically. Manual synthesis often suffers from low efficiency, whereas automatic synthesis requires automatic synthesizers, which are typically very expensive.

Thus, there is a need for a method of high-efficiency peptide synthesis which is simpler, more flexible, and less expensive than using existing automatic synthesizers.

### BRIEF SUMMARY OF THE INVENTION

The invention is directed to an apparatus for semi-automated parallel synthesis of multiple macromolecules, wherein the apparatus comprises at least two liquid reservoirs each in liquid communication with at least one multi-channel liquid dispenser having an array of n nozzles, an array of n reaction containers containing solid substrates, wherein each nozzle is positioned above or adjacent a different reaction container, and at least one base chamber connected to the array of reaction containers for receiving waste from the n reaction containers, wherein n is an integer from two to about 100.

A method for semi-automated parallel solid phase peptide synthesis comprises adding amino acid reagents and non-amino acid reagents to a plurality of reaction containers, each containing a substrate for the synthesis, and removing non-amino reagents from the reaction containers, wherein the amino acid reagents are added manually and the non-amino acid reagents are added and removed using an automated apparatus.

In a method for solid-phase peptide synthesis which method comprises steps of providing an activated polymer resin to a reaction container; performing a first washing step comprising adding a washing liquid to the reaction container to swell the polymer resin and removing the washing liquid to yield a swelled, activated polymer resin; performing a deprotecting step comprising adding a deprotecting liquid to the reaction container, mixing the swelled, activated polymer resin with the deprotecting liquid, and removing the deprotecting liquid from the reaction container; performing a second washing step three times, wherein the second washing step comprises adding a washing liquid to the reaction container and removing the washing liquid from the reaction container; performing a coupling step comprising adding a protected amino acid in a liquid to the reaction container and allowing the amino acid to react; repeating the second washing step; repeating the deprotecting, second washing, coupling, and second washing steps, wherein each coupling step comprises adding a new amino acid to couple to the previously added amino acid; removing the resin from the reaction container; and cleaving the peptide from the resin;

the improvement comprising performing the first and second washing steps and the deprotecting step using an automated apparatus and performing the adding steps manually.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 is a schematic representation of a high through-put peptide synthesis apparatus according to an embodiment of the present invention;

FIG. 2 is a schematic representation of a multi-channel liquid dispenser according to an embodiment of the invention; and

FIG. 3 is a schematic representation of a reaction container according to an embodiment of the invention.

### DETAILED DESCRIPTION OF THE INVENTION

Unless defined otherwise, all technical and scientific terms used herein have the same meaning as is commonly understood by one of skill in the art to which this invention belongs. All publications and patents referred to herein are incorporated by reference.

The invention relates to a semi-automated method for the parallel synthesis of multiple peptides or other macromolecules and a semi-automated, high through-put peptide synthesizer apparatus.

As described in more detail below, the semi-automated method for parallel peptide synthesis according to the invention is based on conventional solid phase peptide synthesis (SPPS) techniques. As in conventional SPPS, the method involves first preparing an activated polymer resin which will act as the solid phase (solid support) on which the peptide synthesis will occur. Subsequently, amino acids are added sequentially to form the desired peptide in stepwise fashion. The first amino acid reacts directly with the activated polymer resin to form a linkage; subsequent amino acids couple to the previously added amino acid(s) to form a growing peptide. After each addition step, washing and deprotecting liquids are added and removed to take away excess (unreacted) amino acid and deprotect the appropriate functional groups, ensuring that the desired peptide bonds will be formed with each subsequently added amino acid. Rather than being a completely manual or a completely automated process, as are known in the art, the method according to the invention involves both manual and automated steps: the addition and removal of washing and deprotecting liquids are performed in an automated manner, whereas the additions of amino acids are performed manually.

### Apparatus

The apparatus for peptide synthesis according to the invention provides for simple, efficient, low-cost, high through-put, semi-automated peptide synthesis, and may be used for building sequence defined peptide chains. It may also be utilized for performing other solid phase reactions known in the art or to be developed. It may be understood that “solid phase reactions” are those that are performed on a solid phase support, such as a polymer resin, typically for the step-by-step growth of a molecule. In addition to peptides, solid phase synthesis may also be used for synthesizing other macromolecules, such as DNA, RNA, and modified oligonucleotides, and in combinatorial chemistry. Accordingly, while the apparatus is described below with respect to the synthesis of peptides, it should be understood that its use is not limited thereto.

The apparatus comprises an array of reaction containers (which are designed to hold the solid supports or substrates for the peptide synthesis (activated polymer resins) and the growing peptides), at least one multi-channel liquid dispenser having an array of nozzles, each positioned above a separate reaction container in the array, two or more liquid reservoirs, each reservoir in liquid communication with the liquid dispenser(s), and at least one base chamber connected to the reaction containers for removing liquid therefrom. In one embodiment, the addition and removal of liquids is controlled by programmable electromagnetic valves. The number of reaction containers in the system may be as large as desired, and is only limited by the practicality of building, maintaining, and controlling the apparatus. Accordingly, the use of a multi-channel liquid dispenser allows for the synthesis of multiple (different) peptides simultaneously in parallel with simple manipulation, requiring minimal bench labor. This represents a significant improvement relative to the conventional manual synthesis which yields only a single peptide at one time.

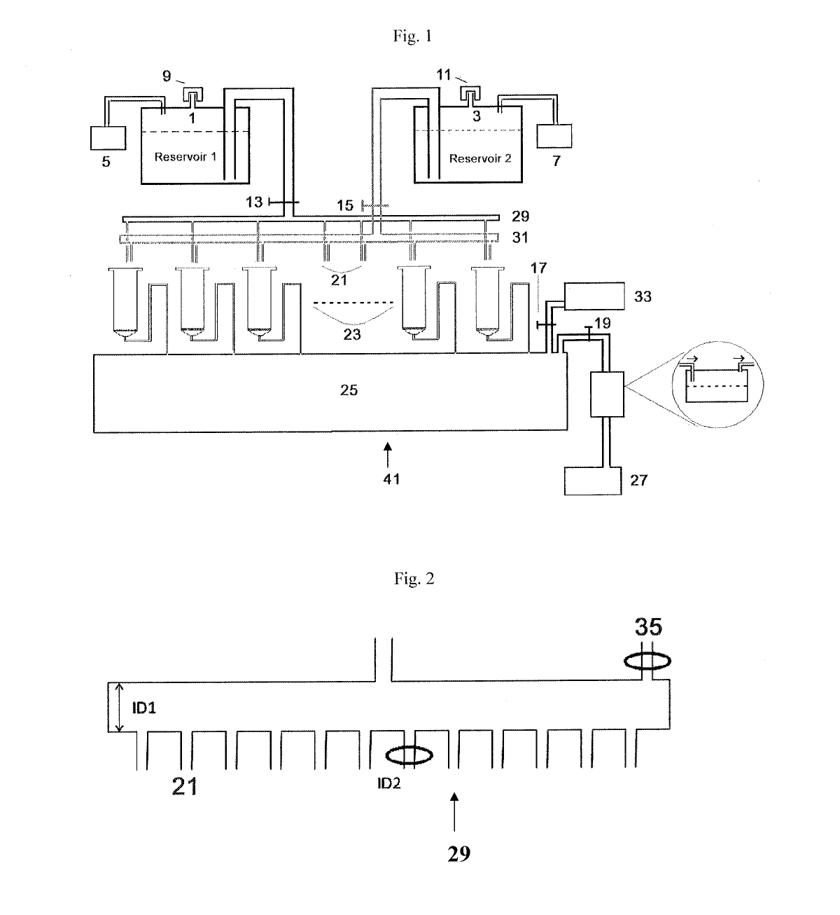

An apparatus according to one embodiment of the invention is shown schematically in FIG. 1, and may be made of glass, plastic, or other suitable laboratory or production materials. The apparatus 41 includes two liquid reservoirs 1 and 3 which are intended to hold and supply liquids (reagents) that are common to all of the reactions that will be performed in the reaction containers. As shown in FIG. 1, reservoirs 1 and 3 contain liquids A and B, respectively.

For example, when used for peptide synthesis, liquid A may be a washing liquid, such as dichloromethane (DCM) or dimethylformamide (DMF). Other washing liquids which are known in the art or to be developed would also be appropriate for use as liquid A. Liquid B may be a deprotecting liquid, for example. Appropriate deprotecting liquids for peptide synthesis are well known in the art and need not be described further; the deprotecting liquid used for a particular synthesis may be determined on a case-by-case basis depending on the protection desired.

Each reservoir is connected to and in liquid communication with a multi-channel liquid dispenser 29, 31. As shown in FIG. 1, each liquid reservoir is connected to a different multi-channel liquid dispenser: reservoir 1 is connected to dispenser 29 and reservoir 3 is connected to dispenser 31, which avoids contamination. However, it is also within the scope of the invention (not shown) to connect both liquid reservoirs to the same liquid dispenser. It is also within the scope of the invention for the apparatus to contain more than two liquid reservoirs (not shown). For example, a third reservoir may contain a third liquid (liquid C), such as a cleavage liquid. Each liquid reservoir is also connected to a pump 5, 7 and a pressure regulator 9, 11, which, along with valves 13, 15, may be controlled by a control (computer) interface (not shown).

The multi-channel liquid dispenser(s) 29, 31 each comprises an array of n nozzles 21, with each nozzle positioned above or adjacent to a reaction container 23 in an array of n reaction containers. It is within the scope of the invention for the number of nozzles and reaction containers (n) to be as few as two to as many as about 100. Preferably, the number of reaction containers is about eight to about sixteen. The liquids in the reservoirs 1, 3 may be transferred automatically and in tandem to the multi-channel liquid dispensers 29, 31 and then to the reaction containers 23 such as by using a pump 5, 7 or a compressed gas such as air, nitrogen, argon, etc. (not shown) and controlled by electromagnetic valves 13, 15. Thus, by utilizing the apparatus, a liquid A or B may be simultaneously added to n reaction containers in an automated fashion.

The reaction containers are designed to contain solid substrates (supports), such as polymer resins, for the solid phase synthesis. The reaction containers have at least one opening for receiving and removing reactants and liquids. Preferably, each reaction container has different openings for receiving and removing reactants and liquids, e.g., at least one opening for receiving and at least one opening for removing. Liquids common to the reactions performed in the reaction containers, such as washing and deprotecting liquids, are introduced to the openings via the multi-channel liquid dispenser, whereas reagents, such as amino acids, are added manually via the same or different openings in the reaction containers. Preferably, reagents and liquids are removed automatically from the reaction containers via the base chamber, as described below. The reaction containers may be open at the top for easy addition or polymer resin and amino acids, but preferably have a top that maybe covered or closed to prevent contamination.

The n reaction containers 23 are also connected to one or more base chamber(s) 25 for receiving waste from the reaction containers. The removal of reaction mixtures or wash liquids from the reaction containers 23 through the base chamber(s) may be controlled by a vacuum pump 33, or by two vacuum pumps 27 and 33, as shown in FIG. 1. The wash liquids that are pumped to the based chamber(s) are sent to waste. The reaction mixtures that are removed through the base chamber(s) may be placed in another container for cleavage from the resin and collection of the peptide products. Alternatively, a cleavage liquid may be added to the reaction containers via the apparatus and the peptide products removed from the reaction containers via the base chamber(s) and then collected. Electromagnetic valves 17, 19 are also used to control the flow of liquid from the reaction containers to the base chamber(s) and from the base chamber(s) for removal from the apparatus. Advantageously, the waste, such as excess washing or deprotecting liquids and excess amino acids, may be removed from the reaction containers simultaneously via the base chamber(s) and then disposed of (or recycled) in a straightforward manner.

In one embodiment, as illustrated in FIG. 2, the multi-channel liquid dispenser 29 comprises an array of nozzles 21 for adding liquids to an array of reaction containers at the same time for high through-put reactions. This configuration provides for the ability to perform the addition of washing and deprotecting liquids simultaneously during the parallel synthesis of different peptides or in other solid state reactions performed in the reaction containers. The dispenser also contains a vent 35 for preventing possible high pressure build-up. The inside diameters of the liquid dispenser 29 (ID1) and of the nozzles 21 (ID2) are important for accurate control of liquid addition. The ratio of ID1 to ID2 is preferably about 20:1 to 20:2 (or 10:1), more preferably about 20:1.3 to 20:1.7. Preferably, ID 1 is about 0.5 mm to 5 mm, more preferably about 1.0 mm to 3.0 mm. These diameters and ratios are preferred to ensure that the liquids are added to the reaction containers in similar amounts at the same time.

The number of reaction containers in the array of reaction containers and the number of nozzles in the array of nozzles is preferably the same, and may be about 2 to about 100, more preferably about eight to about sixteen. The apparatus in FIG. 1 contains an array of n reaction containers, only five of which are shown.

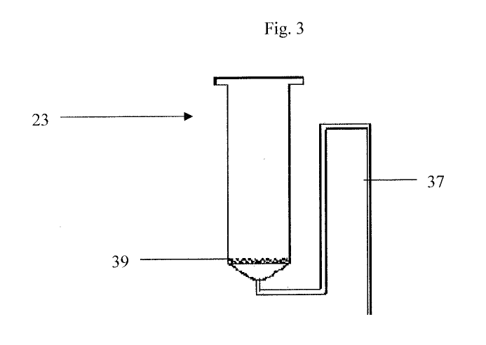

A preferred reaction container is shown schematically in FIG. 3. In this embodiment, the reaction container contains an inverted U-shaped tube 37 to avoid leakage of reaction mixture or liquid from the reaction container. The reaction container also contains a magnetic bar or magnetic stirring device 39 for stirring the reaction mixture. It is also within the scope of the invention to pump compressed gases, such as air, nitrogen, argon, etc., from the base chamber into the reaction container, such as with pumps 27, 33 to make bubbles for mixing the reaction mixture. If it is desired to cleave the peptide from the resin in a separate vessel, a solution may be added to the reaction mixture in the reaction container and then transferred to another container or vessel using a pipet. Alternatively, cleavage liquid may be added to the reaction mixture in the reaction container and then the peptide removed from the reaction container via the base chamber(s). A valve (not shown) may also be connected to the bottom of the reaction container to prevent the leakage of reaction mixture or solvent from the reaction container.

It is within the scope of the invention to utilize a control (computer) interface to monitor and/or control the functions of the valves, pumps, and pressure regulators. The control interface may be programmed to realize automated or semi-automated parallel simultaneous synthesis of multiple peptides. In contrast with prior art apparatuses for peptide synthesis, the apparatus according to the invention is low cost and offers increased flexibility. The addition and removal of liquids are controlled by pumps and programmable electromagnetic valves, thus reducing the cost of the apparatus (commercial automated machines typically cost tens of thousands of dollars) and increasing the efficiency of the peptide synthesis relative to manual synthesis. Another advantage of the apparatus is the flexibility in scale, providing the ability to synthesize peptides on a small or large scale as desired.

### Method of Peptide Synthesis

The semi-automated method for parallel peptide synthesis according to the invention is based on conventional solid phase peptide synthesis techniques. However, rather than being a completely manual or a completely automated process, as are known in the art, the method involves both manual and automated steps. Use of the apparatus described above described above provides for an efficient, simple, semi-automated synthetic process.

### Polymer Preparation

The first step of the method involves providing activated polymer resin(s) to reaction container(s). The number of reaction containers may be two or more, depending on the number of peptides which are to be synthesized in parallel. Methods for preparing activated polymer resins and appropriate resins for SPPS are well known in the art and need not be described. The resins are provided manually to the reaction container(s) which are open at the top or have an easily removable cover for access. The amount of resin may be determined based on the desired scale of the reaction.

In a second step, washing liquid (solvent or solution), such as dimethylformamide (DMF), is added to the reaction container(s) to swell the resin; the DMF is then removed. This step, as well as all subsequent washing steps, is an automated step. Specifically, DMF is present in one reservoir of the apparatus of the invention and is added to the reaction container(s) via the multi-channel liquid dispenser. DMF is then removed from the reaction containers via the base chamber. The washing liquid is not limited to DMF and other washing liquids known in the art or to be developed, such as dichloromethane (DCM), would also be appropriate. The introduction and removal of the washing liquid to and from the reaction containers is controlled by valves and pumps, preferably using a computer or control interface.

### Deprotecting Step

The third step of the method involves adding a deprotecting liquid to the reaction container(s) containing the swelled activated resin(s). This step is automated and is performed using the apparatus of the invention by adding the deprotecting liquid from the second reservoir to the reaction containers via the multi-channel liquid dispenser. The reaction of resin with deprotecting liquid is allowed to proceed for at least about five minutes with mixing, such as with a magnetic stirring apparatus present in the reaction containers. The length of mixing will be understood by one skilled in the art to depend on the particular reaction to be carried out and the nature and volume of the reactants. The deprotecting liquid is then removed from the reaction containers via the base chamber using pumps and valves controlled by a computer interface. Appropriate deprotecting liquids are well known in the art and need not be described further.

### Washing Step

Subsequently, a washing liquid, such as DMF, is added to the reaction container(s) to wash the resin and remove excess deprotecting liquid. The washing liquid is then removed. This automated step, as previously described, is preferably performed three times. The washing liquid is preferably the same liquid as used to swell the resin in a previous step.

### Amino Acid Addition/Coupling Steps

In a fifth step, a first amino acid, preferably dissolved in a solvent such as DMF, is added manually to each reaction container, such as by syringe, pipette, etc. As well known in the art, the amino acid is protected to avoid the formation of random polymer. The amino acids added to each reaction container may be the same or different, depending on the peptides which are desired. Since the particular amino acid added may be different for each reaction container, this is a manual or non-automated step. In this first coupling step, the amino acid will react with the activated polymer resin to form a linkage. The reaction is allowed to proceed with mixing until complete and is preferably monitored to determine when it has been completed.

### Washing Step

Subsequently, an automated washing step as previously described is performed (for example, three times) to remove excess unreacted amino acid from the reaction container(s).

The polymer resin is now coupled to a single amino acid. In order to form a dipeptide, the deprotecting, washing, addition/coupling, and washing steps are repeated so that a further newly added amino acid couples to the amino acid attached to the polymer resin. Subsequently, in order to increase the length of the dipeptide to produce the desired peptide, the deprotecting, washing, addition/coupling, and washing steps are repeated as many times as necessary. In each coupling step, a further newly added amino acid couples to the previously formed intermediated peptide attached to the polymer resin and forms a new peptide bond.

### Removal of Peptide and Purification

Finally, the resin is removed from the reaction container as previously described for cleavage of the peptide and the crude peptide is purified, such as with HPLC. The liquids remaining in the reaction containers are removed, such as via the base chamber of the apparatus.

The method steps described herein are well known in the art for the synthesis of peptides via SPPS. However, known methods for peptide synthesis using this protocol are either all performed manually or all using an automated device, and are both slow and time consuming or extremely expensive. The method according to the invention includes both manual and automated steps, thus providing a low cost method for preparing peptides.

The method for semi-automated parallel solid phase peptide synthesis according to the invention may also be understood to be a method which involves adding amino acid reagents and non-amino acid reagents to a reaction container containing a substrate for the synthesis and removing non-amino acid reagents from the reaction container. In the method, the amino acid reagents are added to the reaction container manually and the non-amino acid reagents are added to and removed from the reaction container using an automated apparatus, such as that described above according to an embodiment of the present apparatus invention.

As explained previously, the method according to the invention has been described in detail with respect to the synthesis of peptides. However, the invention is not limited to peptides, and may also be appropriate for the synthesis of other macromolecules such as DNA, RNA, and modified oligonucleotides, which are synthesized from nucleoside building blocks rather than from amino acids.

Various embodiments of the invention have now been described. It is to be noted, however, that this description of these specific embodiments is merely illustrative of the principles underlying the inventive concept. It is therefore contemplated that various modifications of the disclosed embodiments will, without departing from the spirit and scope of the invention, be apparent to persons skilled in the art.

The following specific example of the invention is further illustrative of the nature of the invention, it needs to be understood that the invention is not limited thereto.

### EXAMPLE

The synthesis of twelve different peptides was performed using an apparatus according to the invention as shown in FIG. 1.

Specifically, peptides with the sequences RAYSPSA, ERDYSPS, CNYYSNS, KIIPFNR, ERTYSPS, NETSYRH, DNYYSNS, ERAYSPS, AMTYPKE, ERTASPS, CKETTMK and ERDYSPS were synthesized simultaneously following the standard solid phase peptide synthesis (SPPS) procedure using the apparatus of the current invention as shown in FIG. 1. 500 mg of 2-chlorotrityl chloride resin (0.2 mmole scale) was added to each reaction container, followed by the addition of 8 ml of anhydrous dichloroform (DCM) to swell the resins. The solvent was removed from the reaction containers after eight minutes, leaving the resins ready for repeated cycles of amino acid coupling. In each cycle, 8 ml of 20% piperidineine in dimethylformamide (DMF) were added to each reaction container at a flow rate of 2 ml/s to remove the protection group for peptide chain elongation. The deprotection was allowed to proceed for 20 minutes and then the deprotection solution was removed using a pump. The resins were washed four times with 8 ml of DMF. Lastly, a mixture of 1.5 mmole of the appropriate Fmoc-protected amino acid and 0.4 ml of diisopropylcarbodiimide (DIC) in DMF was added to the reaction container and the coupling reaction was allowed to proceed for 1 hour. After the coupling reaction, the mixture was removed using a pump and the resin was washed four times with 8 ml of DMF to complete the cycle. The syntheses were completed after all the amino acids were coupled in the correct sequence one at a time. MS analysis confirmed that all twelve peptides were synthesized correctly, having purities in the crude products based on HPLC analysis of 85.37%, 84.32%, 69.95%, 68.36%, 73.21%, 54.32%, 84.46%, 81.21%, 78.17%, 32.98%, 61.51% and 79.15%, respectively.

Various modifications and variations of the described subject matter will be apparent to those skilled in the art without departing from the scope and spirit of the invention. Although the invention has been described in connection with specific embodiments, it should be understood that the invention as claimed should not be unduly limited to these embodiments. Indeed, various modifications for carrying out the invention are obvious to those skilled in the art and are intended to be within the scope of the following claims.

We claim:

1. An apparatus for semi-automated parallel synthesis of multiple macromolecules, wherein the apparatus comprises at least two liquid reservoirs each in liquid communication with at least one multi-channel liquid dispenser having an array of n nozzles, an array of n reaction containers containing solid substrates, wherein each nozzle is positioned above or adjacent a different reaction container, and at least one base chamber connected to the array of reaction containers for receiving waste from the n reaction containers, wherein n is an integer from two to about 100.

2. The apparatus according to claim 1, wherein the solid substrates are polymer resins.

3. The apparatus according to claim 1, wherein the macromolecules are selected from the group consisting of peptides, DNA, RNA, and modified oligonucleotides.

4. The apparatus according to claim 1, wherein each of the at least two liquid reservoirs contains a reagent common to the synthesis of the multiple macromolecules.

5. The apparatus according to claim 4, wherein the reagent is selected from the group consisting of a washing liquid and a deprotecting liquid.

6. The apparatus according to claim 1, wherein each reaction container comprises a first opening for receiving a reactant or a liquid and a second opening for removal of the reactant or the liquid.

7. The apparatus according to claim 6, wherein the reactant is selected from the group consisting of an amino acid and a nucleoside.

8. The apparatus according to claim 1, wherein the at least two liquid reservoirs are coupled to the at least one multi-channel liquid dispenser by an electromagnetic valve.

9. The apparatus according to claim 1, wherein a ratio of an inside diameter of the at least one multi-channel dispenser (ID1) to an inside diameter of the nozzles (ID2) is about 20:1 to 20:2.

10. The apparatus according to claim 1, wherein an inside diameter of the nozzle (ID2) is about 0.5 mm to 5 mm.

11. The apparatus according to claim 5, wherein the inside diameter is about 1.0 mm to 3.0 mm.

12. The apparatus according to claim 1, wherein each reaction container comprises an inverted U-shaped tube for preventing leakage of reaction mixture or solvent from the reaction container.

13. The apparatus according to claim 1, wherein each reaction container comprises a stirring device.

14. The apparatus according to claim 1, further comprising a control interface for automated transfer of liquids from the liquid reservoirs to the at least one liquid dispenser and to the reaction containers and from the reaction containers to the at least one base chamber.

15. A method for semi-automated parallel solid phase peptide synthesis comprising adding amino acid reagents and non-amino acid reagents to a plurality of reaction containers, each containing a substrate for the synthesis, and removing non-amino reagents from the reaction containers, wherein the amino acid reagents are added manually and the non-amino acid reagents are added and removed using an automated apparatus.

16. In a method for solid-phase peptide synthesis which method comprises steps of providing an activated polymer resin to a reaction container; performing a first washing step comprising adding a washing liquid to the reaction container to swell the polymer resin and removing the washing liquid to yield a swelled, activated polymer resin; performing a deprotecting step comprising adding a deprotecting liquid to the reaction container, mixing the swelled, activated polymer resin with the deprotecting liquid, and removing the deprotecting liquid from the reaction container; performing a second washing step three times, wherein the second washing step comprises adding a washing liquid to the reaction container and removing the washing liquid from the reaction container; performing a coupling step comprising adding a protected amino acid in a liquid to the reaction container and allowing the amino acid to react; repeating the second washing step; repeating the deprotecting, second washing, coupling, and second washing steps, wherein each coupling step comprises adding a new amino acid to couple to the previously added amino acid; removing the resin from the reaction container; and cleaving the peptide from the resin;
the improvement comprising performing the first and second washing steps and the deprotecting step using an automated apparatus and performing the adding steps manually.
